(19) United States
(12) Patent Application Publication
(10) Pub. No.: US 2021/0015749 A1
(43) Pub. Date: Jan. 21, 2021

(54) NANOCOCHLEATE FORMULATION AND METHOD OF PREPARING THE NANOCOCHLEATE FORMULATION

(71) Applicants: Ramin Goudarzi, San Jose, CA (US); Maryam Eskandary Nassab, Tehran (IR); Ahmad Reza Dehpour, Tehran (IR); Alireza Partoazar, Tehran (IR)

(72) Inventors: Ramin Goudarzi, San Jose, CA (US); Maryam Eskandary Nassab, Tehran (IR); Ahmad Reza Dehpour, Tehran (IR); Alireza Partoazar, Tehran (IR)

(21) Appl. No.: 16/516,493

(22) Filed: Jul. 19, 2019

(51) Int. Cl. A61K 9/127; A61K 47/24; A61K 47/14; A61K 33/24; A61K 45/06; A61K 31/663; A61K 47/10; A61K 31/593

(52) U.S. Cl. CPC A61K 9/1274; A61K 47/24; A61K 47/14; A61K 33/24; A61K 31/593; A61K 31/663; A61K 9/1277; A61K 47/10; A61K 45/06

(57) ABSTRACT

The present invention discloses a nanocochleate formulation and a method for preparing the nanocochleate formulation. The formulation provides a pharmaceutical composition based on a nanocochleate consisting of phospholipids of phosphatidylserine (PS), cholesterol, at least one drug, and a surfactant, which are consequently stabilized in presence of cations like cerium from degradation agents. The formulation is homogenized to produce nanocochleate containing the drug. The formulation using nanocochleate containing phosphatidylserine and drug such as alendronate or vitamin D as an anti-osteoporotic agent provide significant protection against bone loss, which would need a minimal or even without any drug to improve osteoporosis in osteoporotic animals.

### BACKGROUND OF THE INVENTION

In the past few decades, considerable attention has been focused on the development of ideal drug delivery system. The ideal drug delivery system should deliver the drug at a rate directed as per the body needs over the period of treatment and it should channelize the active ingredient to the site of action. At present, no available drug delivery system behaves ideally. However, lipid-based delivery system such as cochleate and nanocochleates have attracted enormous attention by researchers to improve drug delivery.

The nanocochleate drug delivery vehicle is based on encapsulating the drug in a multilayered, lipid crystal matrix or a cochleate to deliver the drug safely and effectively. Nanocochleates are cylindrical microstructures that consist of series of lipid bilayers. They have unique multilayered structure consisting of a solid, lipid bilayer sheet rolled up in a spiral or in a stacked sheet in order to minimize their interaction with water.

Thus, cochleates have the ability to encapsulate hydrophilic as well as hydrophobic moieties of any shape and size thus making them the most versatile carrier for delivery of a wide range of drug molecules, proteins and peptides. They also protect the entrapped molecule from harsh conditions of pH, temperature and lipase degradation. Owing to this unique structure, cochleates have a potential in encapsulating and delivering small molecule drugs for various diseases such as bone disorders, particularly, osteoporosis.

Osteoporosis is a systemic skeletal disease, characterized by low bone mass and deterioration of bone tissue, with a consequent increase in bone fragility and susceptibility to fracture. Alendronate as the first choice is used against osteoporosis. However, long-term usage of alendronate produces many adverse effects such as gastroesophageal irritation, osteonecrosis of the jaw (ONJ), severe suppression of bone turnover, and prostate cancer and multiple myeloma.

Although cochleate formulations have been developed and proved efficient for several drug deliveries, their formulation process, structural features and parameters influencing drug release are not fully explored. Therefore, there is a need for a method for providing ultrafine nanoparticles from nanocochleate that would be desirable to make an improved drug delivery system rather than cochleates. Further, there is need for a formulation of nanocochleate for drug delivery and synergistic effects. Further, there is a need for a method for preparing ultrafine nanocochleate for treatment of one or more disorders, particularly, bone disorders such as osteoporosis.

### SUMMARY OF THE INVENTION

A formulation and a method for preparing the same is disclosed. In one embodiment, the nanocochleate formulation comprises a negatively charged phospholipid phosphatidylserine, cholesterol, a multipotential agent, at least one drug, and a surfactant. In one embodiment, the nanocochleate formulation for treating one or more disease or disorder, particularly, bone disorder comprises a phospholipid of phosphatidylserine, phosphatidylcholine, an anti-osteoporotic agent, a multipotential agent, and a surfactant.

In one embodiment, the multipotential agent is cerium cation. In one embodiment, the drug is selected from the group consisting any one of antimicrobial drug, antiviral drug, anesthetic and analegesic drugs, anticancer drug, immunosuppressant drug, antiproliferative agent, mTOR inhibitor, steroidal anti-inflammatory agent, non-steroidal anti-inflammatory drug, vasodilatory agent, antiosteoporotic agent, and herbal drug. In another embodiment, the drug is an anti-osteoporotic agent.

In yet another embodiment, the drug is selected from the group consisting any one of tacrolimus and cyclosporine, strontium valerate, alendronate, adriamycin, cabamazepine, melphalan, nifedipine, indomethacin, estrogens, testosterones, steroids, phenytoin, ergotamines, cannabinoids, rapamycin, propanid d, propofol, alphadione, echinomycine, miconazole nitrate, teniposide, taxol, and taxotere.

In yet another embodiment, the drug is selected from the group consisting any one of aminoglycosides, clofazimine and streptomycin, amphotericin B, Ketoconazole, Isoniazid, Rifampicin, acyclovir, amantadine, tenofovir, disoproxil, fumarate, procaine, amethocaine, lidocaine, prilocaine barbiturates, thiopental, benzodiazepines, ketamine, and propofol, cyclophosphamide, methotrexate, 5fluorouracil, doxorubicin, cyclophosphamide, docetaxel, bleomycin, vinblastine, dacarbazine, mustine, vincristine, procarbazine, vincristine, etoposide, cisplatin, epirubicin, capecitabine, folinic acid, oxaliplatin calcineurin inhibitors, mycophenolate mofetil, mycophenolate sodium, azathioprine sirolimus; steroidal anti-inflammatory agents Prednisone; aspirin, ibuprofen, naproxen, celecoxibdiclofenac, diflunisal, etodolac, ibuprofen, indomethacin, tranquilizer, adenosine, amyl nitrite and other nitrites, capsaicin, ethanol, glyceryl trinitrate, sildenafil, tadalafil, vardenafil, tetrahydrocannabinol, strontium valerate, alendronate, risedronate, zoledronic acid, denosumab, avocado, soybean, curcumin and Timolol.

In one embodiment, the surfactant is selected from the group consisting any one of non-ionic surfactant, ionic surfactant, zwitterionic surfactant, medicinal surfactant, biological surfactant, natural surfactant, two-component surfactant, biosurfactant and gemini surfactant.

In another embodiment, the surfactant is selected from the group consisting any one of sorbitan ester of fatty acid and their ethoxylated derivatives, polyol esters, polyoxyethylene esters, poloxamers, polyol esters glycol, glycerol esters, sorbitan derivatives, carboxylates, sulfates, alcohols, phospholipids of phosphatidylserine, phosphatidylethanolamine, phosphatidylcholine, sphingomyelins, phospholipids, fatty acids, lipopeptide and lipoprotein, fiber two glycolipids, bile salts, and gemini surfactants.

In one embodiment, the formulation is prepared in a form selected from a group consisting anyone of cream, gel, lotion, ointment, foam, suppository, spray, capsules, cachets, pills, tablets, lozenges, powders, granules, solution, suspension, emulsion, enema, sterile isotonic aqueous or nonaqueous solutions, and dispersions.

In one embodiment, the formulation is administrable by at least one of an oral administration, topical application, transdermal application, and parental administration.

In one embodiment, a method for producing nanocochleate formulation is disclosed. The method comprises a step of: mixing one or more phospholipids, cholesterol and one or more drugs, wherein the phospholipids comprises phosphatidylserine. The method further comprises a step of: dissolving the mixture in a solvent at a predetermined temperature depending on the melting point of phospholipids. The method further comprises a step of: adding a non-polar surfactant to the mixture.

The method further comprises a step of: hydrating the mixture by double distilled water containing water soluble carbohydrate to form a suspension medium. The step of hydration is done by stirring the mixture at 700-1500 rpm to liposome and removing an alcoholic content from the mixture through evaporation. The method further comprises a step of: homogenizing the suspension medium with a mechanical homogenizer to form a liposome suspension. The method further comprises a step of: adding a solution containing at least one of a trivalent or divalent cation to liposome suspension.

The method further comprises a step of homogenizing the liposome suspension with the mechanical homogenizer to form a nanocochleate suspension. The method further comprises a step of: homogenizing the nanocochleate suspension using an emulsifier shaft to form ultrafine cochleate.

One aspect of the present disclosure is directed to a nanocochleate formulation comprising: phosphatidylserine; cholesterol; a multipotential agent; at least one drug, and a surfactant. In one embodiment, the multipotential agent is cerium cation and the drug is an anti-osteoporotic agent. In one embodiment, the cholesterol is replaced with a phosphatidylcholine.

Another aspect of the present disclosure is directed to a cochleate formulation for treating bone disorder comprising: a phospholipid of phosphatidylserine; cholesterol; an anti-osteoporotic agent; a multipotential agent, and a surfactant. In one embodiment, the anti-osteoporotic agent is alendronate, and the multipotential agent is a cerium cation.

Another aspect of the present disclosure is directed to a method for producing nanocochleate formulation comprising the steps of: (a) mixing one or more phospholipids, cholesterol and one or more drugs, wherein the phospholipids comprises phosphatidylserine; (b) dissolving the mixture in a solvent at a predetermined temperature depending on the melting point of phospholipids; (c) adding a non-polar surfactant to the mixture; (d) hydrating the mixture by double distilled water containing water soluble carbohydrate to form a suspension medium, comprising the step of: stirring the mixture at 700-1500 rpm to liposome and removing an alcoholic content from the mixture through evaporation; (e) homogenizing the suspension medium with a mechanical homogenizer to form a liposome suspension; (f) adding a solution containing at least one of a trivalent or divalent cation to the liposome suspension; (g) homogenizing the liposome suspension with the mechanical homogenizer to form a nanocochleate suspension; and (h) homogenizing the nanocochleate suspension using an emulsifier shaft to form ultrafine cochleate. The solvent is, in one example, pure ethanol, and the non-polar surfactant may be propylene glycol.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

FIG. 1 illustrates a flowchart of a method for preparing nanocochleate formulation, according to an embodiment of the present invention;

FIG. 2 exemplarily illustrates a nanocochleate and ultrafine nanocochleate preparation method, according to an embodiment of the present invention;

FIG. 3A exemplarily illustrates a graph of size distribution of nanocochleate using DLS, according to an embodiment of the present invention;

FIG. 3B exemplarily illustrates a graph of size distribution of ultrafine nanocochleate using DLS, according to an embodiment of the present invention;

FIG. 4A exemplarily illustrates a cochleate image obtained using a scanning electron microscopy (SEM) system, according to an embodiment of the present invention;

FIG. 4B exemplarily illustrates an ultrafine nanocochleate particle image obtained using a scanning electron microscopy (SEM) system, according to an embodiment of the present invention;

FIG. 4C exemplarily illustrates a nanocochleate image obtained using a scanning electron microscopy (SEM) system, according to an embodiment of the present invention;

FIG. 5 is a graph of drug release profile of nanocochleate, according to an embodiment of the present invention;

FIG. 6 is a graph of vitamin D level in the rat serum after 21 days of NanoCOC group administration, according to an embodiment of the present invention;

FIG. 7 exemplarily illustrates the sections of histomorphometric indices related to femur bone in trabecular and cortical examination in NanoCOC-D and non-treatment groups of osteoporosis, according to an embodiment of the present invention.

### DETAILED DESCRIPTION

A description of embodiments of the present invention will now be given with reference to the figures. It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

The present invention also relates to methods for preparing ultrafine nanocochleate for treatment of one or more disorders, particularly, bone disorders such as osteoporosis using nanocochleate containing phosphatidylserine and drug such as alendronate or vitamin D as an anti-osteoporotic agent.

The present invention discloses a nanocochleate formulation and a method for preparing the nanocochleate formulation for drug delivery and synergistic effects. The present invention generally relates to a lipid-based cochleate delivery system, and more particularly relates to a nanocochleate formulation and method of preparing the nanocochleate formulation.

According to the present invention, the formulation provides a pharmaceutical composition based on a nanocochleate consisting of a negatively charged phospholipid phosphatidylserine, cholesterol, a multipotential agent, at least one drug, and a surfactant, which are consequently stabilized in presence of cations like cerium from degradation agents. The formulation is homogenized to produce nanocochleate containing the drug. The nanocochleate drug delivery system is based upon encapsulating drugs in a multilayered, lipid crystal matrix (a cochleate) to potentially deliver the drug safely and effectively.

In one embodiment, the nanocochleate formulation comprises a phospholipid of phosphatidylserine, phosphatidylcholine, a multipotential agent, at least one drug and a surfactant. In one embodiment, the nanocochleate formulation for treating one or more disease or disorder, particularly, bone disorder comprises a phospholipid of phosphatidylserine, a phosphatidylcholine, an anti-osteoporotic agent, a multipotential agent, and a surfactant. In one embodiment, the multipotential agent is cerium cation. In one embodiment, the anti-osteoporotic agent is alendronate.

In one embodiment, the drug is selected from a group including, but not limited to, antimicrobial drug, antiviral drug, anesthetic and analegesic drugs, anticancer drug, immunosuppressant drug, antiproliferative agent, mTOR inhibitor, steroidal anti-inflammatory agent, non-steroidal anti-inflammatory drug, vasodilatory agent, antiosteoporotic agent, and herbal drug. In another embodiment, the drug is an anti-osteoporotic agent. In yet another embodiment, the drug is selected from a group including, but not limited to, tacrolimus and cyclosporine, strontium valerate, alendronate, adriamycin, cabamazepine, melphalan, nifedipine, indomethacin, estrogens, testosterones, steroids, phenytoin, ergotamines, cannabinoids, rapamycin, propanid d, propofol, alphadione, echinomycine, miconazole nitrate, teniposide, taxol, and taxotere.

In one embodiment, the antimicrobial drug is selected from a group including, but not limited to, aminoglycoside, clofazimine, streptomycin, amphotericin B and ketoconazole, isoniazid, rifampicin or combination thereof In one embodiment, the antiviral drug is selected from a group including, but not limited to, acyclovir, amantadine, tenofovir, disoproxil, and fumarate. In one embodiment, the anesthetic and analgesic drugs is selected from a group including, but not limited to, procaine, amethocaine, lidocaine, prilocaine, barbiturate, thiopental, benzodiazepines, ketamine, and propofol.

In one embodiment, the anticancer drug is selected from a group including, but not limited to, cyclophosphamide, methotrexate, 5fluorouracil, doxorubicin, cyclophosphamide, docetaxel, bleomycin, vinblastine, dacarbazine, mustine, vincristine, procarbazine, vincristine, etoposide, cisplatin, epirubicin, capecitabine, folinic acid and oxaliplatin. In one embodiment, the immunosuppressant drug includes, but not limited to, calcineurin inhibitors such as tacrolimus and cyclosporine.

In one embodiment, the antiproliferative agent is selected from a group, including but not limited to mycophenolate mofetil, mycophenolate sodium and azathioprine. In one embodiment, mTOR inhibitor includes, but not limited to, sirolimus. In one embodiment, steroidal anti-inflammatory agent includes, but not limited to, prednisone. In one embodiment, the non-steroidal anti-inflammatory drug includes, but not limited to, aspirin, ibuprofen, naproxen, celecoxib (Celebrex) diclofenac, diflunisal, etodolac, ibuprofen, indomethacin), and a tranquilizer such as phenothiazines, thioxanthines, butyrophenones and clozapine.

In one embodiment, the vasodilatory agent includes, but not limited to, adenosine, amyl nitrite and other nitrites, capsaicin, ethanol, glyceryl trinitrate, sildenafil, tadalafil, vardenafil and tetrahydrocannabinol (THC). In one embodiment, the antiosteoporotic agent includes, but not limited to, strontium valerate, alendronate, risedronate, zoledronic acid and denosumab. In one embodiment, the herbal drug includes, but not limited to, avocado, soybean, curcumin and timolol.

In one embodiment, the surfactant is selected from the group including, but not limited to, non-ionic surfactant, ionic surfactant, zwitterionic surfactant, medicinal surfactant, biological surfactant, natural surfactant, two-component surfactant, biosurfactant and gemini surfactant. In one embodiment, the non-ionic surfactant includes, but not limited to, fatty acid esters of sorbitan, and their ethoxylated derivatives, polyol esters, polyoxyethylene esters and poloxamers. In one embodiment, polyol ester includes glycol and glycerol esters and sorbitan derivatives. In one embodiment, the ionic surfactant includes, but not limited to, carboxylates such as alkyl carboxylates-fatty acid salt, carboxylate fluoro surfactant; sulfates such as alkyl sulfates, sodium lauryl sulfate; and alcehols such as ethanol and propylene glycol.

In one embodiment, the zwitterionic surfactants comprises both cationic and anionic centers such as the phospholipids phosphatidylserine, phosphatidylethanolamine, phosphatidylcholine, and sphingomyelins. In one embodiment, the drugs have surface-active properties such as the antihistamines and the tricyclic depressants, diazepam, chlorpromazine, haloperidol. In one embodiment, the biosurfactants includes, but not limited to, phospholipids, fatty acids, lipopeptide and lipoprotein, fiber two glycolipids and bile salt. In one embodiment, Gemini surfactants (GS) are comprised of two surfactant monomers chemically bonded at or near the headgroups by a rigid or flexible spacer.

In one embodiment, the formulation is applicable for at least one of oral administration, transdermal or tropical application and parental administration. In one embodiment, the formulation is prepared in one or more form selected from a group consisting of cream, gel, lotion, ointment, foam, suppository, spray, capsule, cachets pill, tablet, lozenge, powder, granule, solution, suspension, emulsion, enema, sterile isotonic aqueous or nonaqueous solutions, and dispersions.

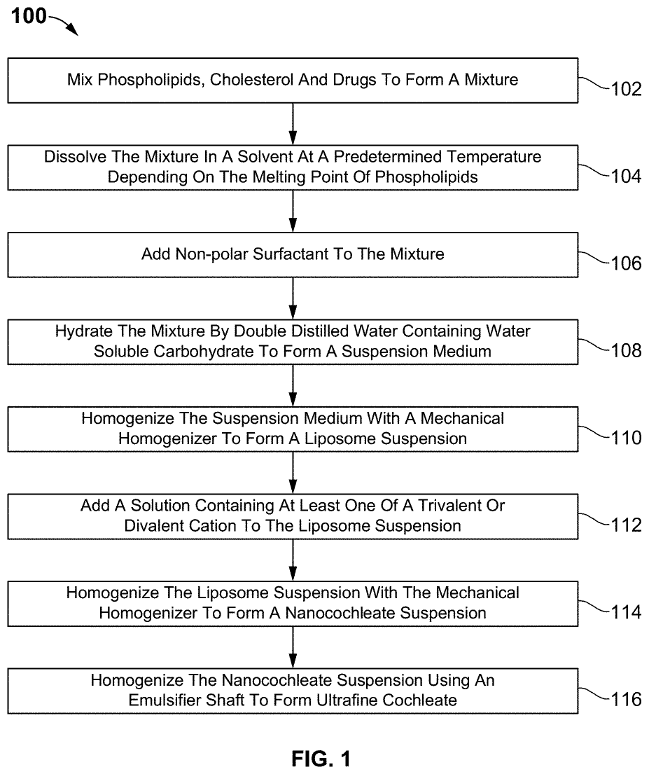

Referring to FIG. 1, the method 100 of preparation of nanocochleate formulation is disclosed. At step 102, an amount of phospholipid, phosphatidylserine, or with other phospholipids and cholesterol, or in combination with a drug such as vitamin D or cyclosporine A are mixed. The phosphatidylserine is higher than phospholipid with a molar percentage. At step 104, the lipid mixture or mixture is dissolved in pure ethanol at 20° C.-60° C. depending on the melting temperature of the phospholipid. The lipid ratio is equal to or greater than the drug as the ratio of 1:1 or 1:0.1, respectively.

The compounds used for drug delivery in the lipid composition include different drug compounds, especially the lipophilic structure, plant protein and peptide compounds, lipid and phospholipid compounds, types of oligonucleotides and human genomes and other compounds of biological or metal and/or non-metallic nanoparticles. At step 106, a non-polar surfactant propylene glycol is added at a rate of 0.1%-30% to the lipid mixture. The amount of the added surfactant depends on the lipid concentration and the nature of the surfactant to the lipid mixture.

At step 108, lipid hydration is carried out by double distilled water, which contains 5%-10% water-soluble carbohydrate solvents such as sucrose or trehalose. The hydration is carried out at the temperature of 20° C.-60° C. In one embodiment, hydration is done at high temperature for phospholipids, such as DPPS and using DOPS the temperature is reduced by 30° C. In another embodiment, the hydration is done by syringe (Nydel 27 or 23 gauge). In yet another embodiment, hydration is done by stirring the mixture at 700-1500 rpm to liposome and finally the alcoholic solvent is removed from the suspension via evaporation. At step 110, the suspension medium is homogenized with mechanical homogenizer for at least 2 minutes to form a liposome suspension.

At step 112, a solution containing trivalent or divalent cations of serenium or cerium or titanium or strontium or calcium at range of 0.1 to 10 mM is added drop wise to liposome suspension, which depends on the amount of phospholipid and used surfactant to fabricate cochleates. At step 114, the suspension cochleate is homogenized with mechanical homogenizer for at least 2 minutes or more to form a nanocochleate suspension at size range of 100-1000 nm. At step 116, the nanocochleate suspension is homogenized using an emulsifier shaft to form ultrafine cochleate.

Advantageously, the cochleate formulation utilizes nontoxic excipients such as propylene glycol. The cochleate is fabricated through duplicated homogenization instead of sonicated procedure, resulting in smaller size and monodisperse nanocochleate formation. The cochleate formulation utilizes cerium cations as a multipotential agent to induce stabilizer, antioxidative and osteocampatiblity in nanocochleate. The present invention replaces homogenization instead of traditional harsh methods of sonication in cochleate preparation. The formulation defines new therapeutic properties of nanocochleate as well as drug delivery to produce synergistic effect with their cargoes for treatment of one or more diseases such as osteoporosis.

Also, the present invention utilizes homogenizer with emulsifier shaft in two stage for making ultrafine structure from cochleates for drug delivery and treatment of one or more diseases, particularly, osteoporosis. The present invention further provides nanocochleate containing phosphatidylserine and an anti-osteoporotic agent such as alendronate for treating bone disorder.

Bisphosphonate such as alendronate is used against osteoporosis to prevent the loss of bone mass. Long term usage of alendronate also produces many adverse effects such as gastroesophageal irritation, osteonecrosis of the jaw (ONJ), severe suppression of bone turnover, and prostate cancer and multiple myeloma. However, according to the present invention, consumption of a three-week nanocochleate containing a low dose of alendronate could cause a significant protection against bone loss via cortical and trabecular bone improvement, and preventing deterioration in chemicho mechanical parameters of osteoporotic in rats. Nanocochleate containing alendronate could provide a considerable osteoporotection effect superior to alendronate alone that suggests a possible dose reduction of the drug, resulting in prevention of its severe side effects and better healthcare and reduction in price of remedy.

Nanocochleate alone could also affect the alleviation of osteoporosis and other severe diseases such as Alzheimer's and transplantation. The ingredients of this formulation of the present invention could be nontoxic and approved by FDA or other organizations.

For example, phosphatidylserine as a dietary supplement that has been recommended for improvement of cognitive functions, also has a novel potentiation in nanocochleate in osteogenesis and bone formation. The safe material and nontoxic agents used in this formulation as well as therapeutic effect of the formulation alone are reasons for performance of this formulation rather than individual drugs.

Furthermore, the procedure of the present invention for nanocochleate fabrication and the nontoxic surfactants produce highly resistant cochleate. Furthermore, duplicating particle homogenization produces monodispersed and ultrafine particles that would be useful for any kind of drug delivery administrations.

One aspect of the present disclosure is directed to a nanocochleate formulation comprising: phosphatidylserine; cholesterol; a multipotential agent; at least one drug, and a surfactant. The multipotential agent may be cerium cation. The drug may be selected from a group consisting of antimicrobial drug, antiviral drug, anesthetic and analegesic drugs, anticancer drug, immunosuppressant drug, antiproliferative agent, mTOR inhibitor, steroidal anti-inflammatory agent, non-steroidal anti-inflammatory drug, vasodilatory agent, antiosteoporotic agent, and herbal drug. In one embodiment, the drug is an anti-osteoporotic agent. The drug may be selected from a group consisting of tacrolimus and cyclosporine, strontium valerate, alendronate, adriamycin, cabamazepine, melphalan, nifedipine, indomethacin, estrogens, testosterones, steroids, phenytoin, ergotamines, cannabinoids, rapamycin, propanid d, propofol, alphadione, echinomycine, miconazole nitrate, teniposide, taxol, and taxotere.

The surfactant may be selected from a group consisting of non-ionic surfactant, ionic surfactant, zwitterionic surfactant, medicinal surfactant, biological surfactant, natural surfactant, two-component surfactant, biosurfactant and gemini surfactant. In another embodiment, the formulation is prepared in a form selected from a group consisting of cream, gel, lotion, ointment, foam, suppository, spray, capsule, cachet, pill, tablet, lozenge, powder, granule, solution, suspension, emulsion, enema, sterile isotonic aqueous or nonaqueous solution and dispersion. The formulation is such that it may be administrable by at least one of an oral administration, topical application, transdermal application, and parental administration. In a related embodiment, the drug is selected from a group consisting of aminoglycosides, clofazimine and streptomycin, amphotericin B, Ketoconazole, Isoniazid, Rifampicin, acyclovir, amantadine, tenofovir, disoproxil, fumarate, procaine, amethocaine, lidocaine, prilocaine barbiturates, thiopental, benzodiazepines, ketamine, and propofol, cyclophosphamide, methotrexate, 5fluorouracil, doxorubicin, cyclophosphamide, docetaxel, bleomycin, vinblastine, dacarbazine, mustine, vincristine, procarbazine, vincristine, etoposide, cisplatin, epirubicin, capecitabine, folinic acid, oxaliplatin calcineurin inhibitors, mycophenolate mofetil, mycophenolate sodium, azathioprine sirolimus, steroidal anti-inflammatory agents prednisone, aspirin, ibuprofen, naproxen, celecoxibdiclofenac, diflunisal, etodolac, ibuprofen, indomethacin, tranquilizer, adenosine, amyl nitrite and other nitrites, capsaicin, ethanol, glyceryl trinitrate, sildenafil, tadalafil, vardenafil, tetrahydrocannabinol, strontium valerate, alendronate, risedronate, zoledronic acid, denosumab, avocado, soybean, curcumin and Timolol.

Another aspect of the present disclosure is directed to a cochleate formulation for treating bone disorder comprising: a phospholipid of phosphatidylserine; cholesterol; an anti-osteoporotic agent; a multipotential agent, and a surfactant. In one embodiment, the anti-osteoporotic agent is alendronate. The multipotential agent may be cerium cation. The formulation may be prepared in a form selected from a group consisting of cream, gel, lotion, ointment, foam, suppository, spray, capsule, cachet, pill, tablet, lozenge, powder, granule, solution, suspension, emulsion, enema, sterile isotonic aqueous or nonaqueous solution and dispersion. In another embodiment, the formulation is administrable by at least one of an oral administration, topical application, transdermal application, and parental administration.

Another aspect of the present disclosure is directed to a method for producing nanocochleate formulation comprising the steps of: (a) mixing one or more phospholipids, cholesterol and one or more drugs, wherein the phospholipids comprises phosphatidylserine; (b) dissolving the mixture in a solvent at a predetermined temperature depending on the melting point of phospholipids; (c) adding a non-polar surfactant to the mixture; (d) hydrating the mixture by double distilled water containing water soluble carbohydrate to form a suspension medium, comprising the step of: stirring the mixture at 700-1500 rpm to liposome and removing an alcoholic content from the mixture through evaporation; (e) homogenizing the suspension medium with a mechanical homogenizer to form a liposome suspension; (f) adding a solution containing at least one of a trivalent or divalent cation to the liposome suspension; (g) homogenizing the liposome suspension with the mechanical homogenizer to form a nanocochleate suspension; and (h) homogenizing the nanocochleate suspension using an emulsifier shaft to form ultrafine cochleate. The solvent may be pure ethanol, and the non-polar surfactant may be propylene glycol. The solution may be at least one of a cerium, titanium, strontium or calcium.

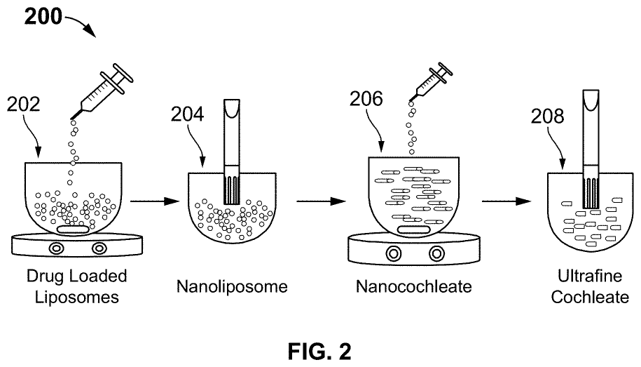

FIG. 2 exemplarily illustrates a nanocochleate and ultrafine nanocochleate preparation method 200, according to an embodiment of the present invention. At step 202, drug loaded liposomes containing surfactant and other stabilizer are prepared through injection method. At step 204, liposome dispersion occurs through homogenizer. At step 206, nanocochleates are formed after adding divalent or trivalent cation solution. At step 208, ultrafine cochleate are formed through homogenization with emulsifier shaft.

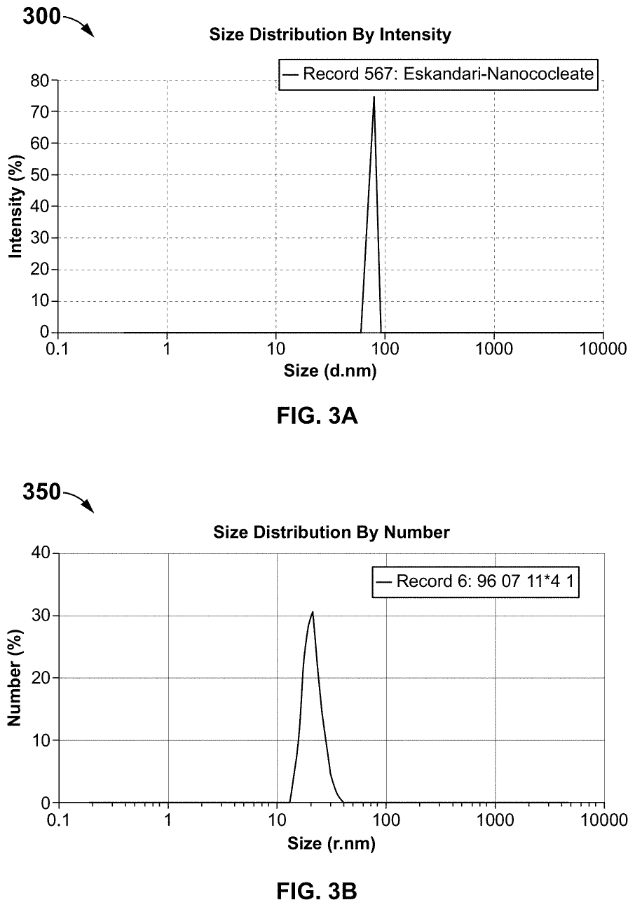

FIG. 3A exemplarily illustrates a graph 300 of size distribution of nanocochleate using DLS, according to an embodiment of the present invention. The size distribution at peak 1 is 76.1 diam. (nm), 100.0% intensity and 4.70 width (nm). The size distribution at peak 2 and peak 3 are 0.00 diam. (nm), 0.0% intensity and 0.00 width (nm). Z-Average (d. nm), Pdl, and intercept are 319, 0.460 and 0.787, respectively.

FIG. 3B exemplarily illustrates a graph 350 of size distribution of ultrafine nanocochleate using DLS, according to an embodiment of the present invention. The size distribution at peak 1 is 141.2 diam. (nm), 1.2% intensity and 39.71 width (nm). The size distribution at peak 2 is 22.44 diam. (nm), 98.8% intensity and 4.285 width (nm). The size distribution at peak 3 are 0.00 diam. (nm), 0.0% intensity and 0.00 width (nm). Z-Average (d. nm), Pdl, and intercept are 141.7, 0.430 and 0.898, respectively.

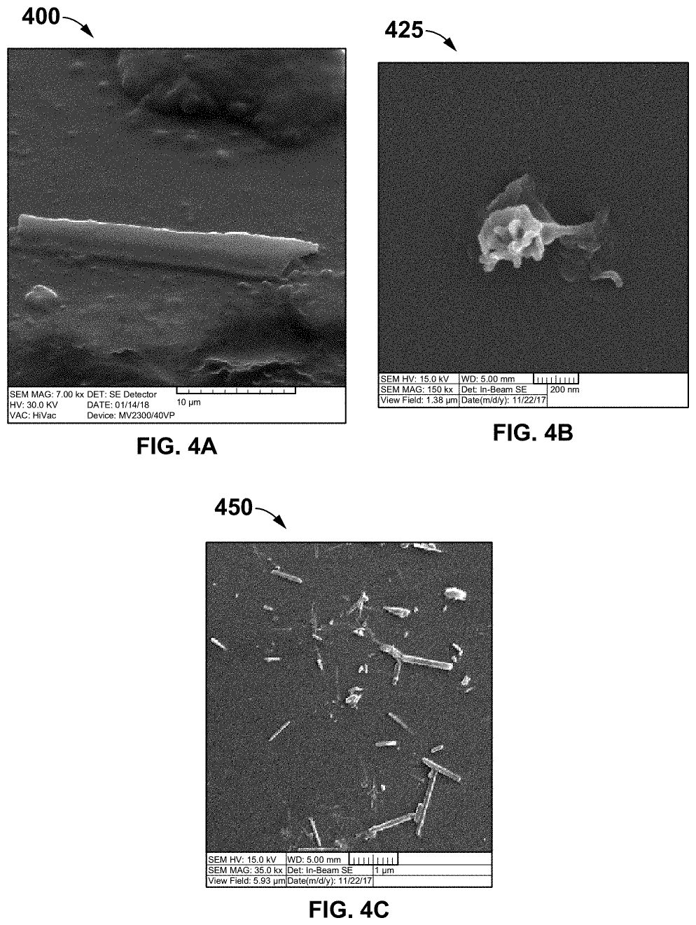

FIG. 4A exemplarily illustrates a cochleate image 400 obtained using a scanning electron microscopy (SEM) system, according to an embodiment of the present invention. FIG. 4B exemplarily illustrates an ultrafine nanocochleate particle image 425 obtained using a scanning electron microscopy (SEM) system, according to an embodiment of the present invention. FIG. 4C exemplarily illustrates a nanocochleate image 450 obtained using a scanning electron microscopy (SEM) system, according to an embodiment of the present invention;

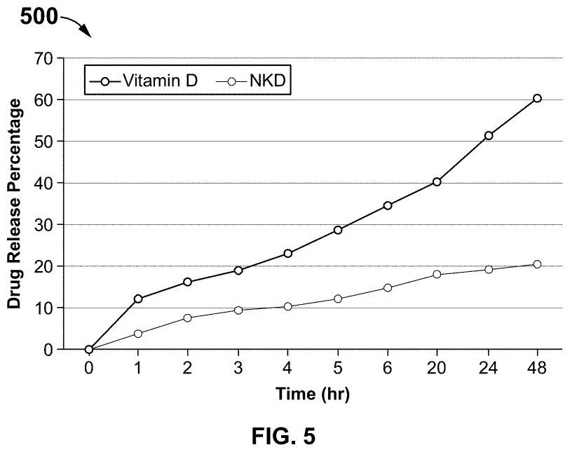

FIG. 5 is a graph 500 of drug release profile of nanocochleate, according to an embodiment of the present invention. According to the graph 500, the release rate of the drug from nanocochleate is about 20% and the free drug is about 60% in the slow-release manner at 48 hours.

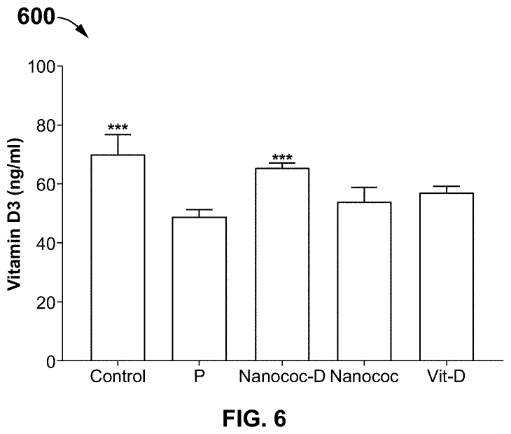

FIG. 6 is a graph 600 of vitamin D level in the rat serum after 21 days of nanococ-D administration, ***; P<0.001 versus prednisolone, according to an embodiment of the present invention. The vitamin D serum subcategories of the samples were divided into groups of: control, prednisolone (P), nanocochleate containing vitamin D (NanoCOC-D), nanocochleate (NanoCOC) and vitamin D (Vit-D). As it shown, the comparison of the groups using ANOVA exhibited that the level of vitamin D serum levels in NanoCOC group was significantly higher than the prednisolone group with respect to its constant dose of vitamin D in each group (P <0.001) as shown in FIG. 6.

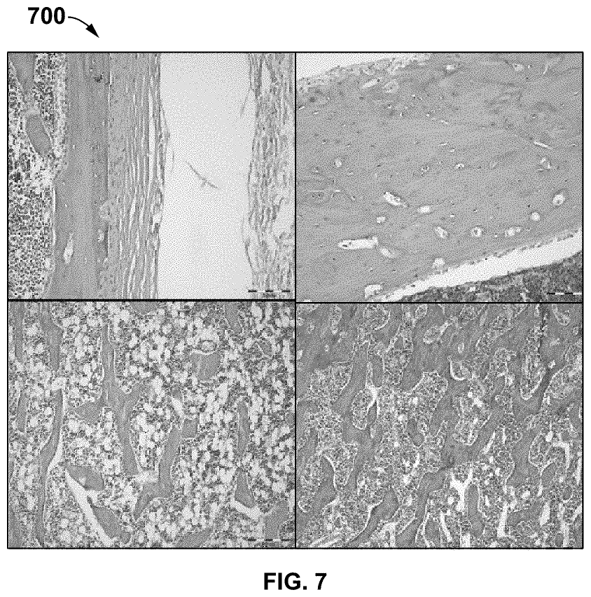

FIG. 7 exemplarily illustrates the sections 700 of histomorphometric indices related to femur bone in trabecular and cortical examination in NanoCOC-D and non-treatment groups of osteoporosis, according to an embodiment of the present invention. A quantitative analysis of bone tissue structure is performed using bone histomorphometric indices that provides valuable information about bone tissue and cellular activity (FIG. 7). Analysis of histomorphometric indices of femoral bone in glucocorticoid-treated rats showed that the number of osteoclasts per square millimeter of the trabecular surface was significantly increased compared with the control group, while the thickness of the cortical bone and its surface area, thickness and trabecular number were increased in Nanococ-D section. According to these data, the effect of NanoCOC-D on the reduction of osteoclasts and, on the other hand, the effect on increased osteoblast are observable, resulting in increasing bone regeneration and preventing bone destruction.

Biochemical tests related to serum parameters of bone in different treatment groups are provided in Table 1.

TABLE 1

| Group | Osteocalcin | Bone-ALP | Ca | Ph | OPG |
|---|---|---|---|---|---|
| Normal | 21.71 ± 3.57**** | 352.8 ± 26* | 9.925 ± 0.3403** | 8 ± 1.283 | 5.04 ± 0.5457*** |
| Prednisolone | 10.04 ± 2.981 | 261.6 ± 57.97 | 9.067 ± 0.1155 | 7.1 ± 0.4583 | 2.483 ± 1.46 |
| (Untreated) | | | | | |
| NanoCOC-D | 24.82 ± 3.623**** | 501 ± 50.92**** | 9.75 ± .3317** | 7.633 ± 1.012 | 5.383 ± 1.348**** |
| NanoCOC | 23.25 ± 1.258**** | 435.5 ± 85.5** | 9.667 ± .05774* | 7.65 ± 0.6191 | 3.81 ± .2475 |
| Vitamin D | 16.5 ± 2.121 | 312 ± 24.42 | 9.367 ± 0.3786 | 7.525 ± 0.4272 | 3.325 ± .9012 |

*Significant difference with osteoporotic group (prednisolone)

Administration of nanocochleate-containing-vitamin D3 and nanocochleate significantly enhanced the serum Ca and Bone-ALP, osteocalcin and OPG levels in osteoporotic treated groups compared with the osteoporotic untreated (P<0.05), whereas the values of serum P (phosphorus) did not show significant differences among the groups. In conclusion, the level of vitamin D in serum by oral administration of nanocochleate loaded vitamin D was significantly improved after 21 day compared with untreated group, as shown in FIG. 6. The beneficial effects of nanocochleate-containing-vitamin D3 are in modulating the majority of bone markers and improving bone tissue structure in osteoporotic animals.

The foregoing description comprise illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one skilled in the art to which this invention pertains having the benefit of the teachings presented in the foregoing descriptions.

Although specific terms may be employed herein, they are used only in generic and descriptive sense and not for purposes of limitation. Accordingly, the present invention is not limited to the specific embodiments illustrated herein. While the above is a complete description of the preferred embodiments of the invention, various alternatives, modifications, and equivalents may be used. Therefore, the above description and the examples should not be taken as limiting the scope of the invention, which is defined by the appended claims.

1. (canceled)

2. (canceled)

3. (canceled)

4. (canceled)

5. (canceled)

6. (canceled)

7. (canceled)

8. (canceled)

9. (canceled)

10. (canceled)

11. A cochleate formulation for treating bone disorder comprising:
a phospholipid of phosphatidylserine;
a phosphatidylcholine;
an anti-osteoporotic agent, wherein the anti-osteoporotic agent is alendronate;
a cerium cation, and
a surfactant, wherein the surfactant is selected from a group consisting of non-ionic surfactant, ionic surfactant, zwitterionic surfactant, medicinal surfactant, biological surfactant, natural surfactant, two-component surfactant, and biosurfactant.

12. (canceled)

13. (canceled)

14. (canceled)

15. (canceled)

16. (canceled)

17. (canceled)

18. A method for producing nanocochleate formulation comprising the steps of:
mixing phosphatidylserine cholesterol and one or more drugs;
dissolving the mixture in an alcohol at a predetermined temperature depending on the melting point of phospholipids;
adding a non-polar surfactant to the mixture;
hydrating the mixture by double distilled water containing water soluble carbohydrate to form a suspension medium, comprising the step of: stirring the mixture at 700-1500 rpm to liposome and removing the alcohol from the mixture through evaporation;
homogenizing the suspension medium with a mechanical homogenizer to form a liposome suspension;
adding a solution containing at least one of a trivalent or divalent cation to the liposome suspension, wherein the solution is at least one of serenium, cerium, titanium, strontium or calcium;
homogenizing the liposome suspension with the mechanical homogenizer to form a nanocochleate suspension, and
homogenizing the nanocochleate suspension using an emulsifier shaft to form ultrafine cochleate.

19. (canceled)

20. (canceled)
